(19) United States
(12) Patent Application Publication
(10) Pub. No.: US 2003/0103964 A1
(43) Pub. Date: Jun. 5, 2003

(54) Methods for designing molecular conjugates and compositions thereof

(76) Inventors: Charles DeLisi, Brookline, MA (US); Jay Berzofsky, Bethesda, MD (US); Kamalakar Gulukota, Westboro, MA (US); Dennis Vac caro, Wellesley, MA (US); Zhiping Weng, Boston, MA (US); Chao Zhang, El Cerrito, CA (US)

Correspondence Address: WEINGARTEN, SCHURGIN, GAGNEBIN & LEBOVICI LLP, TEN POST OFFICE SQUARE, BOSTON, MA, 02109, US

(21) Appl. No.: 10/133,210

(22) Filed: Sep. 5, 2002

Related U.S. Application Data
(63) Continuation of application No. 09/052,530, filed on Mar. 31, 1998, now abandoned.

(51) Int. Cl. A61K 39/395; C12Q 1/70

(52) U.S. Cl. 424/130.1; 435/69.3; 435/320.1; 435/325; 435/5; 530/388.1

(57) ABSTRACT

Improved methods for designing molecular conjugate therapeutics are described. Antibodies are described having specificity for a targeting antigen, said antigen comprising one or more MHC-binding peptides bound to a corresponding class I MHC molecule. When linked to a label or toxic agent, the resulting antibody conjugate can be used for diagnosis, imaging and for treatment against pathogens.

### FIELD OF THE INVENTION

This invention provides improved methods for designing molecular conjugates and compositions thereof, and in particular methods for designing pathogen-directed molecular conjugates for diagnosis and treatment.

### BACKGROUND OF INVENTION

Antibodies have long been thought of as a potential treatment against pathogens and their toxins. Early on, passive immunization with polyclonal antibody was explored as a therapeutic approach to certain diseases. The low titers and lack of specificity of these reagents, however, caused antibody therapy to fall into some disfavor.

The development of monoclonal antibodies (“MAbs” or “MoAbs”) in the late 1970s and early 1980s renewed the hope that antibodies would provide effective therapeutic agents to combat infection. Indeed, early work seemed promising. For example, it was shown that the Fab fragments of a monoclonal antibody directed against the surface coat of malaria sporozoites is active in protecting mice against malarial infection, indicating that Mabs could block attachment of sporozoites to host receptor cells. See P. Potocnjak et al., J. Exp. Med., 151:1504 (1980). Unfortunately, it later became apparent that such antibodies provided only limited protection. For example, while it was found that MAbs infused early after Group B streptococcal infection achieved some benefit, no long-term benefit was evident. See e.g. Christensen et al., Pediatric Res., 18:1093 (1984).

With antibodies showing only partial success, therapy against pathogens has largely depended on drugs. Antibacterial drugs have been directed to inhibition of cell wall synthesis, inhibition of cell membrane function, inhibition of protein synthesis, inhibition of nucleic acid synthesis, and interference with intermediary metabolism. (See e.g., W. K. Joklik et al., [eds.], Zinsser Microbiology, 18th ed., Appleton-Century-Crofts, Norwalk, Conn., [1984], p. 193). Antiviral drugs have been directed to different targets. One approach has involved the use of competing proteins or parts of proteins to block the binding or “fusion” event. An example of this approach can be found in U.S. Pat. No. 4,880,779 which describes inhibitory peptides to block retroviral fusion. Another approach has come from the recognition that some viruses use unique polymerases to replicate nucleic acid. This approach involves the use of competing, nucleotide derivatives to bind to the polymerase and stop replication. An example of this approach can be found in U.S. Pat. No. 4,916,122 which describes synthetic deoxyuridine derivatives to block retroviral nucleic acid replication.

Regardless of the type of pathogen and the particular strategy, drug therapy against pathogens has the drawback that it involves systemic administration of a drug that is potentially toxic to the patient. Since it is necessary to attain certain levels of the drug in the blood in order to provide the proper concentration of the drug at the site of infection, this frequently requires high doses. Thus, to achieve the desired pathogen toxicity, the treatment results in unacceptably adverse side-effects to the patient.

It was recognized that one approach to avoiding high doses and adverse side-effects of drugs was to combine them with antibodies (and in particular, monoclonal antibodies). See e.g., U.S. Pat. No. 4,867,973 to Goers et al. (1989). In this manner, it was thought that the specificity of antibodies could direct the potentially toxic drug to the proper target. Unfortunately, the mutation rate of pathogens (particularly viral pathogens such as HIV) has hampered the development of specific and reliable targeting.

A need therefore exists for a method of identifying and designing better therapeutics. Such an approach should display reliability, high efficiency and an enhanced therapeutic index to permit more effective diagnosis and/or treatment of pathogen infection.

### SUMMARY OF THE INVENTION

The present invention provides improved methods for designing molecular conjugates and compositions thereof, for diagnostics and treatment, including but not limited to the treatment against pathogens of known genomic sequence. In one embodiment, the present invention describes a method for developing pathogen killing agents, including but not limited to antiviral and antimicrobials.

To overcome the problem of high mutation rates, the present invention contemplates targeting by using as the targeting vehicle an antibody conjugate that specifically binds to one or more conserved epitopes of the pathogen antigen complexed to the MHC molecule. Indeed, the method mitigates against the development of viral resistance to drugs and to the immune response, as well as provides a solution for targeting toxic compounds to destroy viruses sequestered in privileged sites which are not easily accessed by cytotoxic T cells. In addition, this method eliminates the virus, whereas current therapies only arrest virus replication.

In one embodiment, the present invention contemplates compositions comprising specific antibodies directed against a target peptide-MHC conjugates, wherein the specific antibody is coupled to a label (e.g. a label useful for imaging), or more preferably, coupled to a cytotoxic agent (e.g., a biological toxin or a radioactive atom).

It is not intended that the present invention be limited to the therapeutic use of such conjugates. Diagnostic and imaging uses are also contemplated. Moreover, when used therapeutically, it is not intended that the present invention be limited to pathogen killing by any one mechanism. It is believed, however, that (at least in some cases) the molecular conjugates of the present invention induce the destruction of the targeted host cell and, thereby, the elimination of the pathogen.

In one embodiment, the invention contemplates therapeutic compositions comprising specific monoclonal antibodies (or portions thereof, such as their Fv domains), directed against a target peptide-MHC conjugate, selected from a human combinatorial library. In another embodiment, the invention contemplates “fusion toxins,” i e. recombinantly produced fusion proteins comprising at least a portion of an antibody and at least a portion of a toxin.

In one embodiment, the invention provides a method of constructing a targeting antigen, said method comprising: a) providing i) sequenced genomes of multiple variants of a pathogen and ii) class I MHC molecules which occur with greatest frequency in a population of interest; b) identifying conserved regions of said genomes, said conserved regions encoding peptides; c) determining which of said peptides bind to said class I MHC molecules, thereby selecting MHC-binding peptides and corresponding class I MHC molecules; and d) selecting a targeting antigen comprising one or more said MHC-binding peptides bound to said corresponding class I MHC molecule.

The targeting antigens provided in the present invention are not limited to a particular type of pathogen. In some embodiments, the said pathogen is a virus, in particular HIV. In other embodiments, the said pathogen is bacterial, fungal or protozoan.

It is not intended that the present invention be limited to only the naturally occurring peptides of a pathogen. In some embodiments, said peptides of the pathogen may be variants of these natural ligands, i e. artificial ligands.

In another embodiment, the invention provides a method of producing an antibody, comprising:a) providing, i) a targeting antigen comprising one or more said MHC-binding peptides bound to said corresponding class I MHC molecule and ii) a host for immunization, b) immunizing said host with said targeting antigen, under conditions such that an antibody is produced, said antibody directed against said targeting antigen. In some embodiments, the specific antibody produced may be a polyclonal antibody, whereas in other embodiments, the antibody produced may be a monoclonal antibody.

In yet another embodiment, the invention provides a method of conjugating an antibody to produce a molecular conjugate, comprising: a) providing, i) antibody directed against a targeting antigen, said targeting antigen comprising one or more MHC-binding peptides bound to a corresponding class I MHC molecule, and ii) a label or a toxic agent; and b) conjugating said label or toxic agent to said antibody under conditions such that a molecular conjugate is produced. In some embodiments, the antibody may be covalently linked to a toxic agent.

It is not intended that the compositions of the present invention are limited to a particular type of toxic agent. In some embodiments, the antibody produced is conjugated to an anti-viral agent. In others, the antibody produced is conjugated to an anti-bacterial agent. In some embodiments, the toxic agent is a biological toxin. In others, the toxic agent is a radioactive atom. In other embodiments, the toxic agent may be selected from the group consisting of antifungals, antineoplastics, radiopharmaceuticals, heavy metals, antimycoplasmals.

### DESCRIPTION OF THE INVENTION

The present invention provides improved methods for designing molecular conjugate therapeutics and compositions thereof, for detection of and treatment against pathogens of known genomic sequence. In general, the present invention describes a method for developing antiviral and/or antimicrobial agent killing drugs. Particularly, it relates to reagents and methods for targeting a diagnostic and/or therapeutic agent to a focus of pathogenic infection by using as the targeting vehicle an antibody conjugate that specifically binds to one or more peptide-MHC complexes, the peptide being encoded by a conserved portion of the nucleic acid of the pathogen (such complexes are hereafter referred to as conserved peptide-MHC complexes). These viral disease-killing molecular conjugates comprise specific antibodies against a target peptide-MHC complex, where the specific antibody is coupled to a cytotoxic agent (e.g., a biological toxin or a radioactive atom). The description of the invention involves the following sections: A) Elements of the Invention, B) Basis for Therapeutic Design, C) Peptide-MHC Selection for Generating Antibody-Conjugates, D) Antibody Generation, E) Mimetics, F) Toxins, G) Radionuclides H) Conjugation of Antibodies to Toxins, I) Radionuclide Labeling.

A. Elements of the Invention

Essentially, the present invention comprises of three basic elements or components, and describes both compositions of matter (e.g., the conjugates themselves) and the method of design of the conjugates. The first component of the present invention comprises the identification of binding ligands suitable as targeting antigens, i.e. antigens to which specific antibodies can be targeted. The procedure comprises (i) finding conserved regions of the pathogen (e.g. viral) genome; (ii) identifying the MHC alleles that are characteristic of a given human subpopulation; (iii) identifying from within the conserved genomic regions, sequences coding for peptides that can bind the class I MHC molecules of the targeted subpopulation. This latter step can be either carried out either computationally as described in U.S. Pat. No. 5,495,423, or by direct experimental binding studies.

The second component of the present invention includes methods of producing antibodies and the resulting antibodies as compositions. The antibodies are made using the above-described targeting antigens and therefore are directed to the above-described targeting antigens. Alternatively, the antibodies can be recombinantly produced antibodies. In some embodiments, antibody-like molecules may be generated to the above-described targeting antigens. These are molecules whose variants have high affinity and high specificity for a large number of targets. These molecules can be constructed from mimetics (discussed below) and may also be recombinantly produced.

In one embodiment, the invention contemplates therapeutic compositions comprising specific antibodies (or portions thereof, such as their Fv domains) directed against a target peptide-MHC complex.

Finally, the third component is the label or toxin conjugated to the above-described antibody. That is to say the antibodies directed against a specific target peptide-MHC complex are coupled to a label or, more preferably, a cytotoxic agent (e.g., a biological toxin or a radioactive atom) to make a molecular conjugate. These molecular conjugates can be used in vitro or in vivo. Moreover, when in vivo use is contemplated, both imaging (e.g. using a conjugate having a label) and pathogen-killing is contemplated. In one embodiment, the invention contemplates the third component in a recombinant form, i.e. “fusion toxins,” i.e. recombinantly produced fusion proteins comprising at least a portion of an antibody and at least a portion of a toxin.

B. Basis for Therapeutic Design

Essentially, the basis of therapeutic design of the present invention is based on the following. It is well known in the art that virtually all nucleated human cells have Class I major histocompatibility complex (MHC) molecules on their surfaces. The number of major class I MHC alleles in the human population is under 100, and each person carries up to six of them drawn from the full population in a non random manner. The class I molecules that they encode continually sample cytosolic protein fragments (8-10 residue long peptides) and present them on the cell surface for surveillance by the immune system. In virally infected cells, those molecules that carry viral peptides are recognized by receptors on cytotoxic T cells, triggering a series of events which kills the infected cell. Because the viral peptide and MHC molecule combine to form a virtually unique molecular moiety, the unwanted side effect of killing healthy cells is minimal. The method of the present invention produces therapeutic conjugates that mimic the immune response to the extent that said pharmaceuticals are directed against peptide-MHC complexes, but are more effective than the immune response because the most important viral escape mechanisms will be severely impeded.

The present invention also solves the problem of overcoming mutation. Because many viruses mutate readily, drugs will be most effective if they are directed against functionally important segments of viral proteins (e.g. portions of the protein which are encoded by regions of the genome that cannot change without destroying the ability of the virus to function—“conserved regions”). A central component of the present invention (discussed above) is the systematic identification of all such conserved regions in pathogens (and in particular, viruses) that constitute a major threat to human health. Identifying conserved regions requires obtaining the genomic sequences of multiple variants of the virus. In general this means sequencing the genome, or gaining available sequenced genomes of the organisms; generating multiple viable strains of the organism; and using computer algorithms or other means known in the art to identify those regions that do not vary from strain to strain. Until recently this, and other details of the method, would not have been viable.

MHC molecules bound to conserved viral peptides are universal molecular targets against which drugs can be directed. More importantly, although there are many such complexes, the set is finite and its members can be delineated. The cells containing these targets can be killed using cytotoxic molecules that bind to them specifically, e.g. monoclonal antibodies conjugated with biological toxins or radioactive atoms.

The present invention also overcomes the problem of sequestration. HIV and other viruses that take refuge in privileged sites will be especially promising targets for this strategy since they are not accessible to T cells, but would be accessible to molecules such as antibody V region domains or their modifications. The present invention exploits both the humoral and cellular aspects of the immune response in a novel way. It takes advantage of the ability of MHC molecules to sample internal proteins, but then uses antibodies, or equally specific molecules, to target infected cells. This has a number of advantages over using cells as the cytotoxic agent, including (a) greater access to tissues; (b) avoiding the need for costimulation and other T cell requirements; (c) avoiding the possibility of viral escape as the result of immunodominance by T cells that react with the variable portion of viral proteins; (d) avoiding the possibility of escape due to gaps in the T cell repertoire; (e) avoiding regulatory mechanisms that turn off T cell responses.

Essentially, the present invention is based on the following information. A virally infected cell typically presents on its surface short viral peptide fragments complexed to class I MHC molecules, which tag the cell for destruction by cytotoxic T lymphocytes. The elucidation of class I MHC structure (Madden et al., 1993, Cell, 75:693-708) and the discovery that bound peptides have MHC-specific sequence motifs (Rammensee et al., 1995, Immunogenetics, 41:178-228) have opened the possibility for modulating the cellular arm of the immune response, the main defense against virally infected cells. In particular, understanding the determinants of binding, through a combination of motifs and free energy calculations (Vajda et al., 1994, Biochemistry, 33:13977-13988), permits searching viral genomes for conserved peptides that will bind stably to any specified MHC product.

The first problem solved by the present invention is to identify the smallest set of MHC alleles, such that a prespecified percentage of the population will carry at least one member of the set. It is of course possible to prepare antibodies against conserved peptide-MHC molecules with representatives from every major MHC allele. This exhaustive strategy would lead to the inclusion of a large number of rare targets. Depending on the health and economic toll of the disease raising antibodies against rare targets may not be economically viable. The present invention demonstrates how to obtain a large degree of coverage at minimal cost. These same considerations for limiting the number of MHC types can also be applied to the development of polyvalent peptide vaccines.

Because HLA alleles are in linkage disequilibrium, the set cannot be identified simply by rank ordering the alleles and choosing those that occur with the highest frequencies. In fact, this so-called set covering problem (Cormen et al., 1990, “Introduction to Algorithms,” McGraw-Hill, New York) belongs to a class of computationally hard problems called NP complete. This simply means that the time required to solve the problem very likely increases as an exponential function of the size of the problem. A reasonably efficient solution can, however, be obtained and is described in the experimental section, example 1 below.

Table 1 (See experimental section, example 2) shows the results of applying the method to class 1 alleles for various of ethnic groups. 90% of most populations can be covered with 5 HLA alleles. The populations that are usually considered homogeneous, such as Japanese, Chinese and Thais, can be covered with fewer alleles. The North American Negroid population turns out to be very diverse; the best possible coverage with 5 alleles is less than 80%. Using 6 alleles, the optimal set is {A2, A3, A23, A28, A30, A33} with a genotypic coverage of 62.2%, corresponding to a phenotypic coverage of 85.7%. Not surprisingly, geographically and historically related populations need similar alleles for majority coverage e.g. compare the Northern and Southern Han Chinese with each other and with Thais, or compare among the European Caucasian populations.

Searches for peptides that bind specific HLA products require either explicit binding studies of candidate peptides, or the examination of binding predictors such as motifs, and free energy considerations. Since population coverage must be among the criteria for selection for HLA molecules for study, Table 1 provides a guide to help prioritize the choice.

Beyond identifying the peptide burden required for the CTL component of a polyvalent vaccine, or for an antibody based therapeutic or diagnostic, and the identification of HLA types which guide the choice of peptide sequences, identification of sets of dominant alleles broadens the basis for establishing correlations between HLA types in a population, and protection from infectious disease (Hill et al., 1991, Nature, 352:595-600). In view of the present invention, allele selection is important and can be applied to wider populations, as high quality frequency and linkage data become available for additional ethnic groups.

C. Peptide-MHC Selection for Generating Antibody-Conjugates

Effective generation of specific antibodies to the target peptide-MHC complex according to the present invention, depends on selecting peptides from the viral genome which satisfy two properties: the peptide is conserved across multiple viral strains, and the peptide binds at least one of the selected MHC alleles. Whether a given peptide satisfies these criteria can be determined by various methods described herein.

Selecting Conserved Peptides: To make specific antibodies to the MHC-peptide complex, the peptides must be expressed peptides selected from proteins encoded by the genome of the virus. This defines the domain of peptides chosen. There are only a few thousand peptides that can possibly be extracted from a virus, and the antibodies will be generated to a few (about 10) from among these. The first step in narrowing the field down is to define “conserved regions” within the viral genome. These are regions that do not show much variation from strain to strain. By what percent does the requirement of conservation reduce the available pool? It is useful to assume about 90% of a set must be identical for conservation. This can potentially reduce the domain to a ½ or ⅓ of the original size (e.g., from 5000 to 2000 peptides). The amount of pruning done in this step differs from virus to virus. The more variable a virus, the smaller its conserved regions and the greater the pruning achieved in this step.

Although, a key component of the strategy is targeting conserved regions, the present invention is not limited to any particular method for locating them. The simplest method is to sequence multiple variants of the genome. One alternative is to solve one or a few three dimensional structures, and identifying those subsequences that are crucial for stability (by e.g., comparing the structures, using free energy functions and what is known about biological function). A preferred method for identifying conserved regions is called “Multiple Sequence Alignment” (MSA). This involves sequencing a large number of different strains and aligning them to each other. This is a standard technique and does not need elaboration, since it is known to the skilled artisan (Also see Taylor et al., J. Mol. Biol. 269: 902, 1997; Gupta et al., J. Comput. Biol. 2: 459, 1995). While it is preferable to select conserved peptides from those encoded by the entire genomic sequence of the target virus, the procedure described herein can be used to select suitable peptides from a smaller set of candidate peptides, such as the set of conserved peptides identified by performing MSA on coat protein amino acid sequences determined for multiple strains of the target virus.

Binding of Peptides to Selected MHC Alleles: Secondly, peptides which bind to particular HLA alleles are selected from the domain of conserved peptides. Determination of the potential for a given peptide to bind to a given HLA allele is a difficult problem, and there are multiple evaluation techniques which address this problem. None of these works perfectly, but the skilled artisan will readily accomplish the selection by successively applying a plurality of the binding evaluation techniques to the domain of conserved peptides. The order in which these evaluation procedures are applied is not critical, although the skilled artisan will usually apply first techniques which most rapidly reduce the number of peptides to be screened in subsequent steps. Suitable evaluation techniques include:

i). Motif Search

A large number of peptides that bind to the given allele are examined for specific sequence patterns. For example, unless certain types of residues occur at certain positions, the peptide will not bind. Such a pattern is called a motif. Any peptide in the domain of conserved peptides that shows this pattern (satisfies the motif) is then predicted to bind and all others are predicted to not bind (See Rammensee et al., Immunogenetics 41: 178-228, 1995). This technique is a very rapid method but its predictions are not completely accurate.

ii). Neural Networks.

A computer program is written to stimulate a “neural network” (NN) [symbol] (Gulukota et al., J Mol. Biol. 267:1258, 1997). NNs are good at extracting and identifying patterns from a sample of data given them. They are “trained” on sequences with known binding status. Then they input the new sequences with unknown binding status (peptides from the conserved regions of the genome). The output state of the NN upon each input indicates whether that peptide binds or not. This method too is rapid and works better than motif searches in that it eliminates many more peptides.

iii). Simple Parametric Models.

Using a model which assigns parameters to different residues occurring in different positions, the corresponding parameters for any new peptide are added up. If the sum exceeds a threshold, the peptide is predicted to bind and vice versa (See Hammer et al., J. Exp. Med. 180: 2353-2358, 1994; Hammer et al., Proc.Natl. Acad.Sci. 91:4456-4460, 1994; Marshall et al., J. Immunol., 154: 5927-5953, 1995; Parker et al., J. Immunol. 152: 163-175, 1994). The parametric methods are also rapid, but again their predictions are imperfect. Methods using parametric models tend to work slightly better than motif searches.

iv). Structure Prediction Followed by Free Energy Evaluation.

This is by far the most detailed computational model. It involves calculating the structure in which the peptide binds the MHC molecule. Given this structure, the free energy of binding is evaluated by using an empirical free energy function, such as that described (See Vajda et al. Biochem 33: 13977, 1994). Structure prediction is computationally very expensive, and therefore less favored when adequate discrimination among the peptides of the conserved peptide domain can be achieved by the other techniques.

v). Experimental Binding Studies.

Experiments for studying the binding of peptides are contemplated by the present invention. Following the synthesis of the peptide, the binding of the peptide in conserved regions to all the alleles chosen can be tested. However, the above methods based on approximate models can be used, if so desired, to reduce the experimental load. Certain peptides are eliminated as unlikely to give any binding, and experiments can then be done only with the rest of the peptides. These methods include but are not limited to (1) binding of peptides to solubilized MHC molecules in detergent, measured by competition for binding of labelled indicator peptides (Buus et al., Science 235: 1353-1358, 1987; Ruppert et al., Cell 74: 929-937, 1993; (2) binding of recombinant MHC molecules to solid phase peptide measured by plasmon resonance (Carr et al., J. Exp. Med. 178: 1877-1892, 1993); (3) stabilization of empty class I MHC molecules on a cell which locks the transporter required for loading endogenous peptides (Nijman et al., Eur. J. Immunol., 23:1215-1219, 1993).

The following procedure provides an efficient method for selecting target peptides for rapidly mutating viruses. The procedure involves 1) selecting a minimum set of HLA alleles that provides sufficient coverage of the target population, (2) determining the binding motif for each of the alleles by using one of the above computational methods to screen the set of conserved peptides; i.e., for each MHC type, eliminate those peptides that are expected to be weak or non-binders, (3) reducing the set of viral peptides by ranking them according to the binding affinity between the peptide and the corresponding HLA allele, and (4) making a cocktail that contains at least one peptide for each of the HLA alleles.

The first step is to examine the HLA allele and haplotype frequencies of the target population, and choose a set of ˜5 alleles such that a large proportion (at least 80%, preferably 90%) of the population is covered. This is an NP-complete problem which can be solved for most ethnic groups using the following algorithm:

1) Pick 5 most frequent A-alleles; calculate coverage of this set

2) Attempt replacing 1,2, . . . 5 of these alleles with B-alleles, such that the coverage is greater.

Any B-allele with frequency less than the least frequent A-allele can be ignored. The same method can be extended to include C-alleles in the set by using C-alleles with frequency in the target population that are greater than the least frequent A-allele already in the set.

Once a set of HLA alleles is chosen, the next step is to determine the binding motifs of the alleles identified in the first step. This may be done experimentally by eluting peptides from the allele, by explicit binding studies, or computationally by mapping the binding site of the allele. The binding motifs provide criteria to reduce the number of possible peptides quickly, facilitating the selection process.

The third step involves selection of suitable peptides. Align sequences of a large number of strains of the pathogenetic virus and delineate the conserved portions. If sequences of a large number of strains are not available but the structure of one strain is available, the components of the structure that are stabilizing can be identified as the likely conserved regions. Search the conserved portions of the viral genome for the motifs identified in step 2. Typically this will lead to a few hundred possible peptide-allele pairings.

The limited set of peptides obtained for the peptide-allele pairings above may then be reduced further as follows. Use dynamic programming or some other suitable algorithm to dock each of the peptide-allele pairs identified above. Calculate their binding free energy and rank order them. Usually, binding rank will be confirmed by experimental binding affinity measurements carried out with the most likely binders, based on their rank order. The method of this invention provides for drastic reduction in the number of experimental measurements necessary to obtain a suitable set of peptides having proven high binding affinity for MHC receptors of particular HLA alleles.

The selected peptide can be refined by some molecular engineering, restricting attention to the residues in the peptide that point toward the MHC, and selecting replacements that will improve the binding affinity. This may be done by calculating the binding affinity of the replacements and rank ordering them as explained above.

A unique feature of this invention is the selection of HLA alleles so as to optimize coverage of the population. This reduces the number of important alleles approximately ten fold. Choosing the alleles with the highest frequency individually will typically lead to suboptimal coverage of the population. Using the algorithm as given in Example 1 increases the coverage typically by about 10%. Also, the search of only the conserved regions enables selection of peptides that will be broadly effective against a large majority of viral strains. Finally, this procedure has lead to reduction of possible peptide candidates by a factor of about 50—from few thousands to about 100.

Once the specific target peptide-MHC complex is determined, the next step comprises generating antibodies to the unique peptide-MHC complex and and, in the case of therapeutics conjugating the antibodies to a toxic agent.

D. Antibody Generation

Both polyclonal and monoclonal antibodies are obtainable by immunization with MHC-peptide complexes or cells bearing these, and either type is utilizable for immunoassays (as well as therapy). Polyclonal sera are readily prepared by injection of a suitable laboratory animal with an effective amount of the purified peptide-MHC complex, collecting serum from the animal, and isolating specific sera by any of the known immunoadsorbent techniques. Antibodies produced by this method are utilizable in virtually any type of immunoassay (see below).

The use of monoclonal antibodies directed to the specific MHC-peptide complexes is particularly preferred because of the ability to produce them in large quantities and the homogeneity of the product. The preparation of hybridoma cell lines for monoclonal antibody production derived by fusing an immortal cell line and lymphocytes sensitized against the immunogenic preparation can be done by techniques which are well known to those who are skilled in the art. (See, for example Douillard and Hoffman, Basic Facts about Hybridomas, in Compendium of Immunology Vol II, ed. by Schwartz, 1981; Kohler and Milstein, Nature 256: 495-499, 1975; European Journal of Immunology 6: 511-519, 1976).

Unlike preparation of polyclonal sera, the choice of animal is dependent on the availability of appropriate immortal lines capable of fusing with lymphocytes. Mouse and rat have been the animals of choice in hybridoma technology and are preferably used. Humans can also be utilized as sources for sensitized lymphocytes if appropriate immortalized human (or nonhuman) cell lines are available. Human antibodies are preferred for treating humans because of the greater in vivo half-life and lower immunogenicity. For the purpose of the present invention, the animal of choice may be injected with an antigenic amount, for example, from about 0.1 mg to about 20 mg of the peptide/MHC molecule complex or antigenic parts thereof. Usually the injecting material is used with an adjuvant (e.g. emulsified in Freund's complete adjuvant). Cells carrying the specific peptide-MHC complex can also be used. Boosting injections may also be required. The detection of antibody production can be carried out by testing the antisera with appropriately labelled antigen. Lymphocytes can be obtained by removing the spleen or lymph nodes of sensitized animals in a sterile fashion and carrying out fusion. Alternatively, lymphocytes can be stimulated or immunized in vitro, as described, for example, in Reading, Journal of Immunological Methods 53: 261-291, 1982.

A number of cell lines suitable for fusion have been developed and the choice of any particular line for hybridization protocols is directed by any one of a number of criteria such as speed, uniformity of growth characteristics, deficiency of its metabolism for a component of the growth medium, and potential for good fusion frequency.

Intraspecies hybrids, particularly between like strains, work better than interspecies fusions. Several cell lines are available, including mutants selected for the loss of ability to secrete myeloma immunoglobulin. The immune cells are best utilized approximately 3 days after the last boost, when they are still activated.

Cell fusion can be induced either by virus, such as Epstein-Barr or Sendai virus, or polyethylene glycol. Polyethylene glycol (PEG) is the most efficacious agent for the fusion of mammalian somatic cells. PEG itself may be toxic for cells and various concentrations should be tested for effects on viability before attempting fusion. The molecular weight range of PEG may be varied from 1000 to 6000. It gives best results when diluted to from about 20% to about 70% (w/w) in saline or serum-free medium. Exposure to PEG at 37° C. for about 30 seconds is preferred in the present case, utilizing murine cells. Extremes of temperature (i.e., about 45° C.) are avoided, and preincubation of each component of the fusion system at 37° C. prior to fusion can be useful. The ratio between lymphocytes and malignant cells is optimized to avoid cell fusion among spleen cells and a range from about 1:1 to about 1:10 is commonly used.

The successfully fused cells can be separated from the myeloma line by any technique known by the art. The most common and preferred method is to choose a malignant line which is Hypoxthanine Guanine Phosphoribosyl Transferase (HGPRT) deficient, which will not grow in an aminopterin-containing medium used to allow only growth of hybrids and which is generally composed of hypoxthanine 1×10⁻⁴M, aminopterin 1×10⁻⁵M, and thymidine 3×10⁻⁵M, commonly known as the HAT medium. The fusion mixture can be grown in the HAT-containing culture medium immediately after the fusion 24 hours later. The feeding schedules usually entail maintenance in HAT medium for two weeks and then feeding with either regular culture medium or hypoxthanine, thymidine-containing medium.

The growing colonies are then tested for the presence of antibodies that recognize the antigenic preparation. Detection of hybridoma antibodies can be performed using an assay where the antigen is bound to a solid support and allowed to react to hybridoma supernatants containing putative antibodies. The presence of antibodies may be detected by “sandwich” techniques using a variety of indicators. Most of the common methods are sufficiently sensitive for use in the range of antibody concentrations secreted during hybrid growth.

Cloning of hybrids can be carried out after 21-23 days of cell growth in selected medium. Cloning can be performed by cell limiting dilution in fluid phase or by directly selecting single cells growing in semi-solid agarose. For limiting dilution, cells suspensions are diluted serially to yield a statistical probability of having only 0.3 cells per well. For the agarose technique, hybrids are seeded in a semi-solid upper layer, over a lower layer containing feeder cells. The colonies from the upper layer may be picked up and eventually transferred to wells.

Antibody-secreting hybrids can be grown in various tissue culture flasks, yielding supernatants with variable concentrations of antibodies. In order to obtain higher concentrations, hybrids may be transferred into animals to obtain inflammatory ascites. Antibody-containing ascites can be harvested 8-12 days after intraperitoneal injection. The ascites contain a higher concentration of antibodies but include both monoclonals and immunoglobulins from the inflammatory ascites. Antibody purification may then be achieved by, for example, affinity chromatography.

Recombinant antibodies are also contemplated, and in particular, single chain antibodies prepared according to Pastan et al., U.S. Pat. No. 5,608,039 (hereby incorporated by reference). In particular, humanized antibodies are contemplated. These can be obtained using phage display libraries or transgenic mice having human antibody genes.

Antibody Assays: A wide range of immunoassay techniques are available as can be seen by reference to U.S. Pat. Nos. 4,016,043 and 4,424,279 and 4,018,653 (herein incorporated by reference). This, of course, includes both single-site and two-site, or “sandwich”, assays of the non-competitive types, as well as in the traditional competitive binding assays.

Sandwich assays are among the most useful and commonly used assays. A number of variations of the sandwich assay technique exist, and all can be used with the antibodies of the present invention. Briefly, in a typical forward assay, a peptide-MHC complex or fixed cell expressing such a complex is immobilized on a solid substrate and the sample to be tested (e.g. hybridoma antibody supernatant) brought into contact with the bound molecule. After a suitable period of incubation, for a period of time sufficient to allow formation of an antibody-antigen secondary complex, a second antibody specific to the antigen, labelled with a reporter molecule capable of producing a detectable signal is then added and incubated, allowing time sufficient for—the formation of a tertiary complex of antigen-antibody-labelled antibody. Any unreacted material is washed away, and the presence of the antigen is determined by observation of a signal produced by the reporter molecule. The results may either be qualitative, by simple observation of the visible signal, or may be quantitated by comparing with a control sample containing known amounts of hapten. Variations on the forward assay include a simultaneous assay, in which both sample and labelled antibody are added simultaneously to the bound antibody. These techniques are well known to those skilled in the art, including any minor variations as will be readily apparent.

In the typical forward sandwich assay, a first antibody having specificity for MHC-peptide complex or antigenic parts thereof, or cell expressing the MHC-peptide complex is either covalently or passively bound to a solid surface. The solid surface is typically glass or a polymer, the most commonly used polymers being cellulose, polyacrylamide, nylon, polystyrene, polyvinyl chloride or polypropylene. The solid supports may be in the form of tubes, beads, discs of microplates, or any other surface suitable for conducting an immunoassay. The binding processes are well-known in the art and generally consist of cross-linking covalently binding or physically adsorbing, the polymer-antibody complex is washed in preparation for the test sample. An aliquot of the sample to be tested is then added to the solid phase complex and incubated at 25° C. (or higher) for a period of time sufficient to allow binding. The incubation period will vary but will generally be in the range of about 1 minute to 2 hours, and more typically 2-40 minutes. Following the incubation period, the antibody subunit solid phase is washed and dried and incubated with a second antibody. The second antibody is linked to a reporter molecule which is used to indicate the binding of the second antibody.

By “reporter molecule” as used in the present specification, is meant a molecule which, by its chemical nature, provides an analytically identifiable signal (e.g. a label) which allows the detection of antigen-bound antibody. Detection may be either qualitative or quantitative. The most commonly used reporter molecules in this type of assay are either enzymes, fluorophores, luminescent molecules or radionuclide containing molecules (i.e. radioisotopes).

In the case of an enzyme immunoassay, an enzyme is conjugated to the second antibody, generally by means of glutaraldehyde or periodate. As will be readily recognized, however, a wide variety of different conjugation techniques exist, which are readily available to the skilled artisan. Commonly used enzymes include horseradish peroxidase, glucose oxidase, beta-galactosidase and alkaline phosphatase, amongst others. The substrates to be used with the specific enzymes are generally chosen for the production, upon hydrolysis by the corresponding enzyme, of a detectable color change. For example, p-nitrophenyl phosphate is suitable for use with alkaline phosphatase conjugates; for peroxidase conjugates, 1,2-phenylenediamine, 5-aminosalicyclic acid, or toluidine are commonly used. It is also possible to employ fluorogenic substrates, which yield a fluorescent product rather than the chromogenic substrates noted above. In all cases, the enzyme-labelled antibody is added to the first antibody-antigen complex, allowed to bind, and then the excess reagent is washed away. A solution containing the appropriate substrate is then added to the tertiary complex of antigen-antibody-antibody. The substrate will react with the enzyme linked to the second antibody, giving a qualitative visual signal, which may be further quantitated, usually spectrophotometrically, to give an indication of the amount of hapten which was present in the sample. “Reporter molecule” also extends to use of cell agglutination or inhibition of agglutination such as red blood cells on latex beads, and the like.

Alternately, fluorescent compounds, such as fluorescein and rhodamine, may be chemically coupled to antibodies without altering their binding capacity. When activated by illumination with light of a particular wavelength, the fluorochrome-labelled antibody adsorbs the light energy, inducing a state to excitability in the molecule, followed by emission of the light at a characteristic color visually detectable with a light microscope.

Immunofluorescent and EIA techniques are both very well established in the art and are particularly preferred for the present method. However, other reporter molecules, such as radioisotope, chemiluminescent or bioluminescent molecules, may also be employed. It will be readily apparent to the skilled technician how to vary the procedure to suit the required purpose.

E. Mimetics

While antibodies can be generated by immunization, the present invention also contemplates antibody-like molecules, that can be constructed from mimetics, as within the scope of this invention. Mimetics are compounds mimicking the necessary conformation for recognition and docking as well as having high affinity and high specificity for a large number of targets. For example, mimetics of antibody-like molecules generated to HIV peptide-MHC complexes are specifically contemplated. A variety of designs for such mimetics are possible. U.S. Pat. No. 5,192,746 to Lobl, et al., U.S. Pat. No. 5,169,862 to Burke, Jr., et al., U.S. Pat. No. 5,539,085 to Bischoff, et al., U.S. Pat. No. 5,576,423 to Aversa, et al., U.S. Pat. No. 5,051,448 to Shashoua, and U.S. Pat. No. 5,559,103 to Gaeta, et al., all hereby incorporated by reference, describe multiple methods for creating such compounds.

Synthesis of nonpeptide compounds that mimic peptide sequences is also known in the art. Eldred, et al., (J. Med. Chem. 37:3882 (1994)) describe nonpeptide antagonists that mimic the Arg-Gly-Asp sequence. Likewise, Ku, et al., (J. Med. Chem. 38:9 (1995)) give further elucidation of the synthesis of a series of such compounds.

The present invention also contemplates synthetic mimicking compounds that are multimeric compounds that repeat relevant peptide sequences. As is known in the art, peptides can be synthesized by linking an amino group to a carboxyl group that has been activated by reaction with a coupling agent, such as dicyclohexylcarbodiimide (DCC). The attack of a free amino group on the activated carboxyl leads to the formation of a peptide bond and the release of dicyclohexylurea. It can be necessary to protect potentially reactive groups other than the amino and carboxyl groups intended to react. For example, the α-amino group of the component containing the activated carboxyl group can be blocked with a tertbutyloxycarbonyl group. This protecting group can be subsequently removed by exposing the peptide to dilute acid, which leaves peptide bonds intact.

With this method, peptides can be readily synthesized by a solid phase method by adding amino acids stepwise to a growing peptide chain that is linked to an insoluble matrix, such as polystyrene beads. The carboxyl-terminal amino acid (with an amino protecting group) of the desired peptide sequence is first anchored to the polystyrene beads. The protecting group of the amino acid is then removed. The next amino acid (with the protecting group) is added with the coupling agent. This is followed by a washing cycle. The cycle is repeated as necessary.

One common methodology for evaluating sequence homology, and more importantly statistically significant similarities, is to use a Monte Carlo analysis using an algorithm written by Lipman and Pearson to obtain a Z value. According to this analysis, a Z value greater than 6 indicates probable significance, and a Z value greater than 10 is considered to be statistically significant. W. R. Pearson and D. J. Lipman, Proc. Natl. Acad. Sci. (USA), 85:2444-2448 (1988); D. J. Lipman and W. R. Pearson, Science, 227:1435-1441 (1985). In the present invention, synthetic antibody-like polypeptides useful in therapy are those peptides with statistically significant sequence homology and similarity (Z value of Lipman and Pearson algorithm in Monte Carlo analysis exceeding 6).

F. Toxins

The toxins which can be conjugated to specific antibodies and are usable herein encompass all toxins used in the production of immunotoxins. Examples of two chain toxins are ricin, abrin, modeccin, diphtheria toxin and viscumin. However, single chain toxins, i.e. toxins composed of A chains only (e.g., gelonin, pseudomonas aeruginosa Exotoxin A, and amanitin) may also be utilized.

Other single chain toxins contemplated include hemitoxins. They include pokeweed antiviral protein (PAP), saporin and momordin. Other useful single chain toxins include the A-chain fragments of the two chain toxins. A chain toxins with multiple B chains such as Shigella toxin are also usable in the invention.

As used herein, 2-chain toxins refers to toxins formed from two chains, and single chain toxins refers to both toxin obtained by cleaving 2-chain toxins as well as toxins having only one chain. A preferred toxin is ricin, a toxic lectin extracted from the seeds of Ricinus communis, which contains an enzymatic and protein synthesis inhibiting A chain and a B chain which contains galactose binding site(s). Ricin is extremely toxic and it has been calculated that a single molecule of ricin in the cytosol will kill a cell.

Ricin may be obtained and purified by the procedures described in U.S. Pat. No. 4,340,535, the disclosure of which is incorporated herein by reference.

G. Radionuclides

Among the radionuclides used, gamma-emitters, positron-emitters, and X-ray emitters are suitable for localization and/or therapy, while beta emitters and alpha-emitters may also be used for therapy. Suitable radionuclides for forming the RIT of the invention include ¹²³I, ¹²⁵I, ¹³⁰I, ¹³¹I, ¹³³I, ¹³⁵I, ⁴⁷Sc, ⁷²As, ⁷²Se, ⁹⁰Y, ⁸⁸Y, ⁹⁷Ru, ¹⁰⁰Pd, ^{101m}Rh, ¹¹⁹Sb, ¹²⁸Ba, ¹⁹⁷Hg, ²¹¹At, ²¹²Bi, ²¹²Pb, ¹⁰⁹Pd, ¹¹¹In, ⁶⁷Ga, ⁶⁸Ga, ⁶⁷Cu, ⁷⁵Br, ⁷⁷Br, ^{99m}Tc, ¹¹C, ¹³N, ¹⁵O and ¹⁸F.

H. Conjugation of Antibodies to Toxins

Conjugation of MoAb to 2 Chain Toxins: For the conjugation of toxins having two chains (such as ricin) to a MoAb, a heterobifunctional cross-linking reagent such as m-Maleimidobenzoyl-N-hydroxysuccinimide-ester (MBS) (Pierce Chemical Company) may be utilized. MoAb (0.5-5.0 mg at a concentration of about 4-6 mg/ml are first reduced with a freshly made solution of 1,4-dithiothreitol (DDT at 0.1 to 1 M) at room temperature for about thirty minutes. The final concentration is 10-100 mM DTT. Approximately 20 minutes following the reduction a freshly made solution of 48 mM MBS and N,N-dimethylformimide (DMF) is added to 1-16 milligrams of the toxin at a concentration of 10-12 milligrams/ml. This gives a final molar ratio of MBS to toxin equal to about 3:1. With ricin, the amount of DMF added should not exceed 10 microliters/ml of ricin in order to prevent denaturation of the protein.

Reduced antibody is purified from the DTT solution by passage through a G-25 superfine column (0.8×4-8 cm) equilibrated in 10 mM Na₂HPO₄.7H₂O, 0.9% NaCl at pH 6.5-7.5. 5-8 drop fractions are collected from the column. A column of that size can effectively desalt a volume equivalent to approximately 15% of the G-25 resin. The absorbance at 280 nanometers is determined and the fractions with the highest readings are pooled. Protein concentration of the pooled fractions is then determined. Recoveries from the G-25 column usuallly range from 50-75% depending on the initial amount of antibody conjugated.

Ricin is added to reduced antibody at a ratio of 7:1 to 18:1. The amount of ricin and antibody are chosen based on the recovery from the G-25 column. The molar ratios of reactants is calculated and the reduced antibody is added directly to the MBS/ricin mixture. The reaction is allowed to proceed at room temperature for 3 hours with occasional stirring.

After 3 hours, the reaction mixture is filtered through a Millipore GV, 0.22 micron filter prior to injection into a TSK SW3000 preparative HPLC column (21.5×600 mm ToyoSoda, Japan). The column is equilibrated in 100 mM Na₂HPO₄.7H₂O, pH 7.2 at a flow rate of 2 ml/min. Antibody characteristically elutes by itself with a peak at 55-60 minutes. A successful conjugation is indicated by a peak of immunotoxin (antibody/ricin conjugate) eluting approximately 2-5 minutes before the antibody peak. Both the immunotoxin and unreacted antibody peaks are collected (approximately 35-45 ml) and stored at 4° centigrade until further purification by affinity chromatography on Sepharose 4B resin. A column of 1×10 cm containing Sepharose 4B is equilibrated in 100 mM Na₂HPO₄.7H₂O, 150 mM NaCl, pH 7.5 at 4° centigrade. Since any material containing ricin will bind to the Sepharose 4B by means of the galactose binding site of ricin B chain, the immunoconjugate binds to the column. The end reactive material is washed through the column by adding 5-10 ml of Na₂HPO₄.7H₂O buffer. The absorbance is monitored at 280 nm and when the absorbance level returns to nearly the base line level, immunotoxin is eluted from the column by washing with buffer containing 50 mM lactose. The product is collected, filter sterilized and kept at 4° centigrade or frozen at −70° centigrade until use.

Conjugation of MoAb to Single Chain Toxins: Radioimmunotoxins can be made utilizing other toxins which are single polypeptide chains. Some, such as hemitoxins, have the advantage of not binding by means of a native receptor to human cells. The conjugation procedure employs the cross-linking reagent N-succinimidyl-3-(2-pyridyldithio)-propionate (SPDP, Pharmacia) which introduces a disulfide bond between the antibody and toxin moiety. The toxin (at least 5 mg/ml concentration) is first incubated with freshly made solution of SPDP (26 mM in N,N-dimethylformamide). The final molar ratio of SPDP to toxin is 3:1. The mixture is incubated for 30 minutes at room temperature with occasional stirring. SPDP modified toxin is purified from the reaction mixture by passage through a G-25 superfine column (0.8×4 cm) equilibrated with 40 mM Na₂HPO₄.7H₂ O, 150 mM NaCl at pH 6.5. The conjugated toxin is concentrated using an Amicon Centricon 10 microconcentrator. The concentration of toxin is determined by absorbance readings at 280 nanometers. The amount of antibody selected to give a final molar ratio of 3:1 toxin to antibody is then reacted for 30 minutes at room temperature with the amount of SPDP (6.5 mM) necessary to give a molar ratio of 3:1 SPDP to antibody. The antibody-SPDP reaction mixture is then passed over a G-25 superfine column equilibrated in 40 mM Na₂HPO₄.7H₂O, 150 millimolar NaCl, at pH 7.5. During this time the SPDP modified toxin is reduced with 100 millimolar DTT at a final concentration of 5 mM DTT in order to release pyridyldithio groups. The reduction proceeds for 30 minutes at room temperature and then the toxin is purified from DTT by passage over a G-25 superfine column in Na₂HPO₄.7H₂O buffer, pH 7.5. The reduced and derivatized toxin is again concentrated using the Centricon 10 device and immediately mixed with SPDP modified antibody to give a final molar ratio of 3:1 toxin to antibody. Molecular weights of 29,000 to 30,000 daltons (toxin); 150,000 daltons (antibody); and 312 (SPDP) are used to calculate molar ratios. The reaction proceeds overnight at 4° C. With occasional stirring before filtration and injection into the TSK SW 3000 preparative HPLC column (equilibrated with 100 mM Na₂HPO₄.7H₂ O, pH 7.2 at a flow rate of 2 ml/minute). Individual fractions are collected and contain mainly immunotoxin or antibody. Since these hemitoxins do not contain B chain binding site, they cannot be further purified on Sepharose 4B resin. The HPLC fractions are, therefore, assayed directly and kept frozen at −70° C. until use. This method is preferred for linking single chain toxins but can also be used for linking two chain toxins.

I. Radionuclide Labeling

The conjugated MoAb-toxin may then be labeled with a radionuclide. Alternatively, either the MoAb or toxin or both may be radiolabeled before conjugation.

Immunotoxin Labeling with Radionuclide: When the radionuclide of the Radioimmunotoxin (RIT) is an iodine isotope, the iodine monochloride micro method described by Contreras, M A; Bale, W F and Spar, I. L. in “Iodine monochloride (IC1) iodinatin techniques,” Methods in Enzymol 92:277-292 (1983) must be utilized in order to create an RIT having a killing activity of 2 logs or greater. The chloramine-T method which has been widely used and described in patents to radiolabeled antibodies has been found to cause a loss of binding activity of the antibody and also a loss of toxicity of the toxin itself. When 0.5 mg aliquots of immunotoxin is labeled with mCi quantities of radioiodine and a 5:1 molar ratio of IC1 to immunotoxin to a specific activity of at least 0.5 mCi/mg, without loss of antibody binding activity or toxin cytotoxicity is obtained.

The iodine monochloride micro method used is described below. The immunotoxin is dialyzed against borate buffer (0.16 M NaCl, 0.2 M H₃BO₃ and 0.04 M NaOH) adjusted to pH 8 with NaOH. This dialysis continues with a minimum of four changes of 20 volumes of the borate buffer, which is essential to remove reducing substances that would otherwise compete with the tyrosine residues for positive iodine. The dialysis tubing is pretreated by heating at 90° centigrade in 0.05% EDTA, pH 7-8 with NaHCO₃ for 10 minutes, then copiously rinsed with distilled water and stored at 4° centigrade in borate buffer. The iodination apparatus is a Reactivial (Pierce) with a triangular matrix teflon coated magnetic stir vane and teflon/silicon cap insert, which is rinsed with borate buffer. The vial with stir vane and protein in place is vented with a tuberculin syringe filled loosely with glass wool. Approximately 1 ml of protein solution at 0.5 mg/ml is used in a 3 ml vial.

The isotope ¹³¹I or ¹²⁵I (Amersham) in the form of iodine ions (NaI) is diluted with borate buffer to 0.15 ml and added to the Reactivial with a 21 gauge needle and disposable syringe. Immediately, the vane is briefly and gently activated to mix protein and isotope. IC1 of an appropriate dilution from 0.02M stock (0.02M IC1, 2.0M NaCl, 0.02M KCl and 1.0M HCl) is made with 0.85% NaCl, 0.015N HCl adjusted to 1 ml to give 5 equivalents of IC1 per immunotoxin molecule. The IC1 is mixed immediately prior to addition to the Reactivial with the vane rotating briskly (avoid foaming). After 4-5 seconds the agitation is stopped and incorporation proceeds for one minute whereupon 1 ml of 5% human serum albumin is added as a protective protein against radiation damage, the contents mixed and the solution withdrawn with a 21 gauge needle and a disposable syringe. The protein solution is then passed over a Dowex 1-X4 resin (50-100 mesh Bio-Rad) ion exchange column of about 3 ml bed volume that has been prerinsed with 0.85% NaCl and 5% human serum albumin. The protein solution is followed by a 1 ml rinse of protective protein and then by 1 ml of 0.85% NaCl giving a final volume of 1.8 times the reaction volume.

The immunotoxin could be labeled with a variety of beta-emitting metallic radionuclides using the bicyclic anhydride of DTPA as the chelating agent. (Hnatowich, D. J. et al. in “Radioactive Labeling of Antibody: A Simple and Efficient Method,” Science 220:613-615 (1983) and Hnatowich, D. J. et al. in “The Preparation of DTPA Coupled Antibodies Radiolabeled with Metallic Radionuclides: An Improved Method,” J. Immunol. Methods, 65:147-157 (1983) describes suitable means which may be used in the radiolabeling of this invention.)

The DTPA is heated with an excess of acetic anhydride in pyridine for 24 hours. The anhydride is collected by filtration and washed repeatedly with acetic anhydride and dry ether. Characterization of the structure of the bicyclic anhydride is confirmed by infrared spectroscopy and melting point.

Coupling of the DTPA anhydride to monoclonal antibody or immunotoxin is carried out as follows: a 0.1 mg/ml solution of the DTPA anhydride in dry chloroform is prepared, and an aliquot containing the desired weight is added to the reaction test tube and evaporated to dryness at room temperature by a flow of nitrogen. A solution of 0.5 mg antibody or immunotoxin buffered at pH 7.0 with 0.05M bicarbonate buffer is added to the solid anhydride (7 μg) for a 7:1 anhydride to protein molar ratio, and the solution is agitated for 1 minute. The coupled antibody is purified from free DTPA by passage through a 5 cm Sephadex G-50 gel filtration column. Fractions (1.0 ml) are collected from the G-50 column.

The fraction containing the highest concentration of protein is labeled with the beta-emitting radionuclide. Labeling is by ligand exchange, accomplished by adding a 0.5M acetate buffer solution of radionuclide to the reaction solution. The reaction vial is agitated for 5 minutes. The radionuclide-acetate solution is prepared by adding an equal volume of 1.0 M acetate to the radionuclide-chloride solution, so that the final pH is 6.0. After the addition of radionuclide to the coupled protein solution, 0.1 ml of 25% human serum albumin is added to protect against radiation damage. The specific activity of the labeled antibodies or immunotoxins following dialysis to separate unbound radionuclide should be at least 0.5 mCi/mg.

Radiolabeling the antibody and toxin after coupling is preferred since there is greater retention of specific cytotoxic activity and a slower elimination of the reagent from the blood pool following intravenous injection. Another advantage is that since both the MoAb and toxin are radiolabeled, each can contribute to the death of malignant cells whether they are present on the cell surface (antibody) or internalized (antibody or toxin). Both the toxin and the antibody can be radiolabeled prior to immunotoxin synthesis. Chelating agents optimized for binding alpha-emitting radionuclides to antibodies have also been developed (Se Zalultsky and Bigner, Acta Oncologica 35: 373, 1996). Other chelating agents known in the art may also be used.

### DETAILED DESCRIPTION OF INVENTION

While this invention is satisfied by embodiments in many different forms, preferred embodiments of the invention are herein described, with the understanding that the present disclosure is to be considered exemplary of the principles of the invention and is not intended to limit the invention to the embodiments illustrated and described.

One embodiment of the method uses an antibody Fv domain against the target peptide MHC complex, selected from a human combinatorial library using, e.g. phage display. (See Reiter et al., Proc. Natl. Acad. Sci., USA 94: 4631, 1997). The domain is then humanized to minimize side effects. The human molecule, or its modifications, can be coupled to a toxin which can be a biological (e.g. ricin), or a radioactive atom; e.g. an alpha emitter such as bismuth-213 and radium-223.

Essentially, the distinguishing features of the present invention are namely: (1) sequencing, or differentially sequencing, the genomes of multiple variants of the organism to find conserved coding regions, (2) finding peptides encoded in those conserved regions, either computationally or experimentally, that bind the MHC of interest; (3) using the peptide-MHC complexes to select molecules such as V region antibody domains that bind them specifically and with high affinity; and (4) using the specific antibody molecules with a cytotoxic agent, such as a toxin.

The full array of conserved peptides that bind each MHC of interest can be identified experimentally and using appropriately automated binding assays and peptide synthesizers. Some subset can also be identified by a non-exhaustive and less expensive procedure by using, for example, computational methods described in U.S. Pat. No. 5,495,423 to screen the conserved regions for binding peptides. Although the correlation between binding affinity and presentation is strong, not all peptides that bind MHC will be presented to T cells. The reason is that a cell's digestive machinery does not cleave proteins into all possible binding fragments. This is not a fundamental problem, since cells from infected patients can be used prior to therapy to obtain tissue type (i.e. MHC alleles) and the sequences of MHC bound peptides. Such information is already available for HIV (See experimental section, example 2 and 3).

Since each Class I MHC molecule will bind a limited number of conserved peptides whose sequences usually differ from one class I type to the next, the number of potential targets that need to be identified will be relatively small. For example, a highly conserved sequence from the HIV-1 p24 protein was found bound to HLA-A2 on cells from patients with AIDS, and 5 highly conserved RT sequences have been found in other infected individuals. HLA-A2 is present in approximately 50% of the U.S. Caucasian population, and is invariably present in no fewer than a third of most human subpopulations. Thus, even drugs directed only against HLA-A2 bearing cells would cover a large percentage of the human population (See experimental section, example 3 for more details).

The identification of conserved regions for much of the HIV genome is immediately possible because the DNA sequences of hundreds of strains have already been obtained. Similarly a pool of human T cell epitopes is available, obviating the need for massively parallel binding studies. For most other viruses, finding conserved regions will require sequencing the genome, generating variants (or using natural variants when they are abundant enough) differentially sequencing the variant (i.e. looking for differences from the original sequence), and testing the conserved regions for binding. Other virus for which a large number of sequence variants is available is human papilloma. HPV types 6 and 11 cause genital warts; types 16 and 18 cause cervical cancer.

The method of the present invention may also apply to microbial agents. The tubercule bacillus and chlamydiae are examples of non viral targets to which it would be applicable. Whatever the organism, the goal is the development of a large number of high affinity antibody like molecules directed against conserved peptide-MHC complexes and their storage—either physically or in the form of information or systems required for their rapid production.

Knowledge of conserved genomic regions will provide a major resource that will undoubtedly be in great demand for drug targets and vaccine development. Even traditional approaches to drug targeting, which seek to find and exploit differences between human and microbial genomes, will want to target the conserved regions of microbial genes.

The method and conjugates thereof have been made possible by (i) the revolution in nucleic acid sequencing technology, which now permits entire genomes to be sequenced relatively rapidly so that conserved regions can be identified. (ii) Combinatorial libraries and phage display technology which permits hundreds of millions of antibodies to be generated, from which high affinity anti peptide-MHC antibodies can be selected. (iii) The structure of MHC peptide complexes which can be used in conjunction with advanced computer software to determine which portions of a genome will bind any given class I MHC molecules.

These present state of the art and the availability of a universal molecular target with a finite and manageable number of variants, provide a new concept in drug discovery and a strategy for a systematic and coherent attack on viral and microbial diseases. Furthermore the main component technologies, although recent, are successfully being used in research laboratories and industry.

Those skilled in the art will recognize the use of the following component technologies as they relate to the present invention:

Phage Display:—Conventional methods for raising antibodies specific for peptide MHC complexes do not generate an adequate sample of the full antibody repertoire; as a result, specific high affinity antibodies have been difficult to develop. Combinatorial libraries of some 10⁸ Ig variable domains are now available for selection of high affinity members by phage display. Such antibodies can be prepared as described in U.S. Pat. No. 5,580,717 and humanized versions of the antibodies can be prepared as described in U.S. Pat. No. 5,565,332, incorporated in total herein by reference, using peptide-MHC complexes selected.

Fusion Toxins—It is well known in the art that a number of fusion toxins are in phase I and phase II clinical trials, predominantly against various forms of cancer. Ricin A chain has been used with murine monoclonal antibodies in phase I clinical trials against small cell lung carcinoma, Hodgkin's disease, and B cell lymphoma. Immunoglobulin fused with blocked ricin B chain has been used in clinical trials against non Hodgkin's lymphoma. Recently, phage display selected antibodies were used to construct a fusion toxin that specifically killed mouse class I MHC cells presenting a hemagglutinin peptide from influenza 14 (See Reiter et al., PNAS 94: 4631-4636, 1997). Such fusion toxins can be prepared as described below (also see U.S. Pat. No. 5,608,039, incorporated herein by reference), and conjugated to the antibodies against the selected peptide-MHC complex.

Preparation of Antibody Fusion Proteins: Once a DNA sequence has been identified that encodes an Fv region which, when expressed shows specific binding activity, fusion proteins comprising that Fv region may be prepared by methods known to one of skill in the art. The Fv region may be fused directly to the effector molecule (e.g. cytotoxin) or may be joined directly to the cytotoxin through a peptide connector. The peptide connector may be present simply to provide space between the targeting moiety and the effector molecule or to facilitate mobility between these regions to enable them to each attain their optimum conformation. The DNA sequence encoding the connector may also provide sequences (such as primer sites or restriction sites) to facilitate cloning or may preserve the reading frame between the sequence encoding the targeting moiety and the sequence encoding the effector molecule. The design of such connector peptides will be well known to those of skill in the art. Thus, for example, Chaudhary et al., Nature, 339: 394-97 (1989); Batra et al., J. Biol. Chem. 265: 15198-15202 (1990); Batra et al., Proc.Natl. Acad. Sci. USA, 86: 8545-8549 (1989); Chaudhary et al., Proc. Natl. Acad. Sci. USA, 87: 1066-1070 (1990), all incorporated by reference, describe the preparation of various single chain antibody-toxin fusion proteins.

Generally producing immunotoxin fusion proteins involves separately preparing the Fv light and heavy chains and DNA encoding any other protein to which they will be fused and recombining the DNA sequences in a plasmid or other vector to form a construct encoding the particular desired fusion protein. However, a simpler approach involves inserting the DNA encoding the particular Fv region into a construct already encoding the desired second protein.

An alternative method for producing human antibodies is to use mice that have human antibody genes. The advantage of this is that mice can be immunized with the antigen of interest. The result will be a combinatorial library whose affinities are relatively high. In particular, the method involves immunizing a mouse with the chosen antigen following prior stimulation of the non-human animal's B cells. Methods of producing human antibodies using mouse transgenics are well known to those of skill in the art, and can be prepared as described in U.S. Pat. Nos. 5,641,488, and 5,545,807 incorporated herein by reference.

Alpha emitters such as bismuth-213 and radium-223 can also be utilized as a component of the viral or microbial killing moiety. The primary advantages of alpha particles, aside from their high energy, is that their decay length is only a few cell diameters. Consequently specificity is excellent. Radiolabels, as opposed to biological toxins, will also stimulate little or no immune response, depending on the method of conjugation. In addition, unlike some biological toxins they will not need to be internalized to be effective. Methods of producing Radioimmunotoxins are well known to those of skill in the art, and can be prepared as described in U.S. Pat. No. 4,831,122, incorporated herein by reference. The use of radiolabels and human antibodies should significantly reduce side effects found using biological toxins.

Accordingly, this invention is not limited to the particular embodiments disclosed, but is intended to cover all modifications that are within the spirit and scope of the invention as defined by the appended claims.

In vitro and in vivo Uses of the Invention

The antibodies and antibody conjugates of the present invention have a variety of uses. Such uses include both in vitro and in vivo applications. This section describe some illustrative in vitro and in vivo applications.

In vitro Applications

i) Localization, Quantitation, and In Situ Detection of Specific Peptide-MHC Class I Complexes: CD8+ T lymphocytes recognize antigens as short peptides bound to MHC class I molecules. Current available methods cannot determine the number and distribution of these ligands on individual cells or detect antigen-presenting cells in tissues. In the present invention, a method is described for eliciting and identifying monoclonal antibodies specific for a particular peptide-MHC class I combination. One such antibody can identify antigen complexes with a limit of detection approaching that of T cells. Antibodies can be used to determine the number of peptide-class I complexes generated upon viral infection, to identify antigen-presenting cells in cell mixtures, to determine the site of peptide-MHC class I interaction inside cells, and to visualize cells bearing specific peptide-MHC class I complexes after in vivo infection. Similar antibodies may prove useful for diagnostic or therapeutic purposes in cancer, infectious diseases, and autoimmune disorders.

Essentially, T lymphocytes do not recognize intact proteins as antigens. Instead, their clonally distributed receptors (T cell receptors [TCRs]) interact with ligands composed of short peptides derived from protein antigen and bound to major histocompatibility (MHC) class I or class II molecules [Yewdell, J. and Bennink, J. “The binary Logic of Antigen Processing and Presentation of T Cells” Cell 62:203-206 (1990)] [Germain, R. “MHC-Dependant Antigen Processing and Peptide Presentation: Providing Ligands for T Lymphocyte Activation.” Cell 76:287-299 (1994)]. This feature of T cell immune recognition has precluded direct tracking of antigen-presenting cells (APCs) in vivo, because analysis of antigenic protein distribution cannot determine whether properly processed peptides derived form this molecule are bound to MHC proteins and expressed at the surface of the identified cells. Direct detection of particular peptide-MHC molecule combinations using flow cytometry or immunohistochemistry would allow quantitation of TCR ligands on individual cells, phenotyping of such APCs, and localization of these APCs within normal or pathologic tissues, while confocal immunofluorescence microscopy would permit analysis of the intracellular site(s) of peptide-MHC molecule interaction and trafficking. In situ localization of APCs bearing particular TCR ligands would be especially valuable in characterizing the cell-cell interactions involved in initiation, propagation, and maintenance of T cell immune responses. Multicolor histochemistry could be used to reveal not only the type and location of APCs but also the phenotype of interacting T cells, including the set of cytokines elicited.

The issues of specificity and detection sensitivity that are central to the utility of any monoclonal antibody (MAb) are especially critical in the case of MAbs specific for peptide-MHC molecule complexes. Lysis by high-affinity specific T cells can require only a small number of (<10-100) of ligands per target [Demotz et al., “The minimal Number of Class II MHC-Antigen Complexes needed for T Cell Activation.” Science 249:1026-1030 (1990); Harding, C. and Unanue, E. “Quantitation of Antigen Presenting Cell MHC Class II/Peptide complexes necessary for T Cell Stimulation.” Nature 346:574-576 (1990); Christinck et al., “Peptide Binding to Class I MHC on Living Cells and Quatitation of Complexes required for CTL Lysis.” Nature 352:67-70 (1991); Sykulev et al., “Evidence that a Single Peptide-MHC Complex on a Target Cell can Elicit a Cytolytic T Cell response.” Immunity 4:565-571 (1996)], each of which also expresses on its plasma membrane as many as 10⁵ or more identical MHC molecules associated with hundreds or thousands of other peptides [Rudensky et al., “Monoclonal Antibody Detection of a Major Self Peptide-MHC Class-II Complex.” J. Immunol 148:3483-3491 (1991); Chicz et al., “Predominant Naturally Processed Peptides Bound to HLA-DR1 are Derived from MHC-Related Molecules and are Heterogeneous in Size.” Nature 358:764-768 (1992); Hunt et al., “Characterization of Peptides Bound to the ClassI MHC Molecule HLA-A2.1 by Mass Spectrometry.” Sciencei 225:1261-1263 (1992)]. Although a few MAbs primarily reacting with particular peptide-MC class II combinations have been reported [Aharoni et al., “Immuno-modulation of Experimental Allergic Encephalomyelitis by Antibodies to the Antigen-la Complex.” Nature 351:147-150 (1991); Murphy et al., “Monoclonal Antibody Detection of a Major Self Peptide-MHC Class II Complex.” J. Immunol 148:3483-3491 (1992); Eastman et al., “A Study of Complexes of Class II Invariant Chain peptide: Major Histocompatability Complex Class II Molecules using a new Complex-Specific Monoclonal Antibody.” Eur. J. Immunol 26:385-393 (1996)], as a rule antibodies to MHC molecules do not discriminate among the individual members of this large population of potential T cell ligands. Some monoclonal anti-MHC class I alloantibodies react with a subset of the molecules encoded by a single allele because they are occupied by diverse peptides sharing structural features (Bluestone et al., “Peptide-Induced Conformational Changes in Class I Heavy Chains Alter Major Histocompatability complex Recognition.” J. Exp. Med. 176:1757-1761 (1992); Catipovic et al., “Major Histocompatibility Complex Conformational Epitopes are Peptide Specific.” J. Exp. Med. 176:1611-1618 (1992); Hogquist et al., “Peptide Variants Reveal How Antibodies recognize Major Histocompatibility Complex Class I.” Eur. J. Immunol 23:3028-3036 (1993)]. Although these latter reagents are more selective in their reactivity, they are not useful for tracking particular peptide-MHC class I molecule combinations on APCs with a physiologically diverse cohort of occupied class I proteins.

Other reported MAbs react with one peptide bound to an MHC class I molecule but not with at least one other peptide bound to the same MHC class I protein in tests using class I molecules devoid of endogenously processed peptides [Duc et al., “Monoclonal Anitbodies Directed Against T Cell Epitopes Presented by Class I MHC Antigens.” Int. Immunol 5:427-431 (1993); Andersen et al., “A Recombinant Antibody with the Antigen-Specific, Major Histocompatibility Complex-Restricted Specificity of T Cells.” Proc Natl. Acad. Sci. USA 93:1820-1824 (1996)]. In the former case, analysis with cells displaying a diverse pool of class I-associated peptides again shows staining, suggesting substantial reactivity with self-peptide-associated class I molecules. Because any antibody that binds to more than a very small number of such self-peptide-MHC class I complexes will be incapable of identifying cells bearing physiologically relevant (i.e., very low) levels of an antigenic peptide-MHC class I combination, this reagent has not proved useful for identifying specific antigen complexes. In the latter case, careful tests of reactivity with cells expressing a broad range of self-peptide complexes have not been reported.

Because of the potential value of MAbs with suitable specificity for T cell-recognized antigens, in the present invention, a screening strategy has been described that permits the production and identification of B cell hybridomas producing MAbs specific for a particular peptide-MHC class I complex.

ii) Diagnostic method for detecting viral infection: Currently it is very difficult, time consuming and expensive to detect the presence of most viral infections. Taking AIDS as an example, the first line of testing is to look for antibody from the host specific for the AIDS virus. This technology does not detect very early infection, before the antibody is generated by the host. False negatives are a potential result of the emergence of new viral subtypes. An indirect test of the disease is done by following the decline of CD4 target cells over time. This measurement is primarily diagnostic for patient health. The major diagnostic test for virus is the measurement of HIV RNA in plasma. This test is done by performing an amplification technology (such as PCR) and is therefore quite complicated and prone to problems. There is no direct measure of the extent of disease.

The present invention can potentially be used for viral diagnosis. By being able to target the viral peptide-MHC complex with a labeled antibody (or fragment), potentially one has the ability to:

a) Measure which cell types are infected.

b) Measure what percentage of a cell type is infected by virus

c) Measure overall disease status in time

d) Distinguish viral clades and subtypes in any one patient

e) Perform the above on currently available instrumentation in hospitals

First, the specific antibody generated to the peptide-MHC complex can be fluorescently-labelled. It could then be used in the same FACS (Fluorescence-Activated Cell Sorter) systems in which CD4 measurements are made. FACS systems can distinguish various cell types in blood. This is done by counting cells as they go through the machine and by measuring the fluorescence of certain standard antibodies which define the various cell sub populations. By reading the fluorescent label on our antibodies (if there is enough sensitivity) such instrumentation can detect the presence of peptide/MHC complex on the surface of cells. Cocktails of antibodies may be needed to increase sensitivity and to define viral types. These FACS systems are routinely available as are experienced personnel. Calculations of which cells are infected and what percentage of the population shows fluorescent label are easily performed.

Alternatively, a simpler system of measurement could be performed with radio, enzyme, luminescent or fluorescent-labeled anti-viral peptide-MHC antibody used in an immunoassay format. This sort of assay could distinguish virus and subtypes and disease status but would not be able to give information on which cells were infected. The assay is performed by incubating the labeled antibody with the cells in question. The cells are then centrifuged and washed. After the cells are cleared of unbound label, the bound label is then measured. Bound label measures the presence of specific virus. Clearly some of the antibodies being developed for therapy would also be valuable for diagnosis. A diagnostic product line would require the eventual production of a large number of antibodies. An analogous situation is the Cluster Differentiation (CD) antibodies for distinguishing cell types. Sequences from a variety of viruses would be obtained as well as from clades and subtypes of a particular virus. Comparisons would be made to find sequences unique to each virus and subtype, such as sequences which are conserved but different from one virus to another, etc. These antibodies could be initially made available as research products (like most CD antibodies). Methods of generating antibodies and assays employing the same are as described in the Experimental and Description sections of the invention.

In vivo Applications

(1) Treatment of Infection: Patients having the relevant viral infection, can be injected i.v. with an appropriate amount, generally 1-50 mg depending on the specific activity of the toxin or radionuclide reagent of a cocktail of antibody-toxin conjugates specific for the relevant peptide-MHC complexes. Several treatments are contemplated to be necessary to eliminate newly infected cells as they start to express viruses [symbol] Of particular importance is antibody affinity (See Alexander-Miller et al., PNAS, USA 93:4102-4107). Therapy is most effective shortly after infection, when concentrations of viral antigens in infected cells are low. However, high affinity antibodies are required to bind targets expressing a low number of peptide-MHC complexes, so affinity may turn out to be a crucial parameter, and methods to increase it are likely to be very important.

(2) Scanning of Organs and Tissues: Labelled peptide-MHC specific antibodies could be used for isotopic scanning to determine tissue or organ sites of infection. Antibodies have been used to target viral proteins, such as the HIV envelope protein, that appears on the surface of infected cells. The advantage of targetting MHC-peptide complexes rather than surface expressed proteins, is the increased availability of targets. The peptides can be from any viral protein, not just those that appear on the surface. Moreover, surface proteins tend to be expressed late. Targetting peptide-MHC complexes opens the possibility of attacking infected cells at an earlier stage of infection.

Experimental

In order to facilitate a more complete understanding of the invention, a number of Examples are provided below. However, the scope of the invention is not limited to specific embodiments disclosed in these Examples, which are for purposes of illustration only.

In some of the examples below, antibodies are discussed. Preferred methods for antibody generation and testing are as follows:

Immunizations, Fusions, and Hybridoma Screening: Subject cells, or cell lines preincubated at 28° C. for 24 to 36 hr, can be incubated with 100 μM of the selected peptide in RPMI-25 mM HEPES for 4-6 hr at 37° C., irradiated (3000 rad), washed, and injected intraperitonally four times into BALB/c (H-2^{d}) mice at 12-14 day intervals, at 1×10⁶ to 5×10⁶ cells per inoculation. Sera are harvested from immunized mice and tested for the presence of antibodies with specific peptide-MHC specificity. Mice producing such antibodies are reboosted, spleens harvested 4 days later, and the splenocytes fused with SP2/0 cells as described (Harlow and Lane, 1988). Growing fusion wells are screened by separately staining vector-pulsed and peptide-pulsed host cells with the supernatants from the wells. Further screening of the positive wells can be performed with peptide-pulsed host cells.

Flow Cytometry: Subject cells are incubated with primary antibody for 30 min at 4° C.; washed with phosphate-buffered saline (PBS)-5% fetal calf serum-0.1% sodium azide; and then incubated with a second antibody for 30 min at 4° C., washed, and resuspended in the same medium plus propidium iodide to exclude dead cells during analysis. Sera can be used at 1:20 to 1:200 dilution and MAb-containing supernatant at 1:1 to 1:10 dilution. The second antibody for the sera and for hybridoma screening was fluorescein isothiocyanate (FITC)-rabbit-anti-mouse immunoglobulin (1:1 to 1:10 dilution, DAKO A/S, Denmark). Stained cells are analyzed using a FACScan flow cytometer (Becton-Dickinson, Mansfield, Mass.).

### EXAMPLE 1

In this example, methods for selecting alleles are described:

Consider a cocktail of K peptides P₁, P₂, . . . , P_{K} which bind to HLA molecules M₁, M₂, . . . , M_{K}, respectively. The efficacy of this cocktail is given by the percentage of the population that has at least one of the HLA molecules M₁, . . . , M_{K}. The problem is to select the smallest number of HLA types needed to cover some specified proportion of a population. Equivalently, one can ask how to maximize coverage of a population using a specified number of HLA types.

The simplest case of this selection problem occurs when one restricts attention to alleles of one locus. In this case, the HLA types under consideration are unlinked, i e., no individual chromosome has more than one of the HLA alleles under consideration. The overall coverage Pₜₒₜ of a chosen K-set is simply the sum of the individual coverages Pᵢ

$$P tot = {\sum }_{i = l}^{i = k} Pi \qquad ( 1 )$$

Therefore the optimal K-set is found simply by choosing alleles with the greatest individual frequencies Pᵢ

When this restriction is removed by considering alleles from multiple loci, one has to take account of linkage between loci, i.e. of cases where two or more of the chosen alleles occur on the same chromosome with correlated frequencies. The overall coverage of a chosen set is then the sum of individual coverages corrected for the overlaps

$$P tot = {\sum }_{i = l}^{i = k} Pi - {\sum }_{all pairs} Pij + {\sum }_{all triplets} Pijk \qquad ( 2 )$$

Pᵢⱼ is the probability of a alleles i and j occurring on the same chromosome, etc. This generalized problem is much harder and involves choosing attributes of individuals that will maximize Pₜₒₜ when the frequencies and overlaps of these attributes are known. Its complexity stems from the fact that HLA alleles are in linkage disequilibrium; i.e., the joint probability of a given allelic pair is usually not equal to the product of their individual probabilities (Pᵢⱼ≠PᵢPⱼ).

The problem is NP complete and only an exhaustive search through all possible K-sets of alleles will guarantee finding the optimal K-set. The present invention uses a type of exhaustive search procedure which allows termination of the search at an early stage in the case of most ethnic groups.

Attention is confined to the class I HLA loci A and B, ignoring C for lack of A/C linkage data. This means that coverage that could be obtained with more complete data, is at least as good as what is found here. Briefly, to choose K alleles of maximal coverage, first choose K alleles with the highest individual frequencies from an A-locus. Next attempt to replace 1,2, . . . K of these alleles with B- or other A-alleles. When replacing 1 of the A-alleles with a B-allele, it is only necessary to consider those B-alleles whose frequency of occurrence is greater than that of the least frequent A-allele chosen. Similar statements can be made about replacing 2 or more alleles except that these replacements can be a mixture of B- and hitherto unchosen A-alleles. Typically, the method truncates the search very early. Thus even-though in the worst case, this procedure will take exponential time, it is efficient in the case of most ethnic groups.

### EXAMPLE 2

In this example, Allele Sets for different populations are described. Preferred allele sets are shown in Table 1. These sets were obtained as follows. Allele and haplotype frequencies were tabulated [See Imanishi et al. “Patterns of nucleotide substitutions inferred from the phylogenies of the class I major histocompatibility genes,” J. Mol. Evol. 35: 196 (1992)] and used to determine 3, 4 and 5 allele lists which maximize converge of the different populations. The algorithm used for this is explained in Example 1. Only A- and B-locus alleles were considered. Since only two loci are under consideration, the target function of eq2 can be truncated after the second term (triplet and higher correlations are exactly 0). Pair correlations were tabulated in Imanishi et al. (1992) only if at least two cases of a given pair were found. For the untabulated cases, the pair correlations were assumed to be zero. When data become available for the ignored pair correlations, the procedure can be reapplied, which may change the optimal K-set, or slightly reduce the coverage for a given K-set.

Ethnic groups for this calculation were chosen rather arbitrarily with two guiding criteria: 1) groups should be representative of the populations in the world and 2) statistics of reasonably good quality should be available. Under the name of each ethnic group, is shown the sample size on the basis of which the frequency statistics were determined in Imanishi et al. (1992). Allele frequencies measure the occurrence of a given gene on one of the 6th pair of chromosomes. This is genotypic coverage. Phenotypic coverage refers to the percentage of people that express a given gene. This differs from genotypic coverage since each person has two sets of HLA loci on the two chromosomes in the 6th pair. The two loci were considered mutually independent and the phenotypic coverage F was estimated from the genotypic coverage G as, F=1−(1−G)²=2G−G².

TABLE 1
HLA choices for optimal coverage of different populations

| Ethnicity | 3 Alleles | 4 Alleles | 5 Alleles |
|---|---|---|---|
| (Sample size) | (G; F) | (G; F) | (G; F)^{a} |
| W. African | A2, A28, A33 | A2, A28, A30, A33 | A2, A28, A30, A33, B35 |
| (negroid) (70) | (48.4; 73.3) | (60.2; 84.2) | (71.3; 91.8) |
| N. American | A2, A30, B53 | A2, A28, A30, B53 | A2, A28, A30, A33, B53 |
| (Negroid) (447) | (39.0; 62.8) | (47.3; 72.2) | (54.8; 79.6)^{b} |
| Albanian | A1, A2, A3 | A1, A2, A3, A24 | A1, A2, A3, A24, A32 |
| (208) | (51.0; 76.0) | (61.1; 84.9) | (69.8; 90.9) |
| British | A1, A2, A3 | A1, A2, A3, B44 | A1, A2, A3, A11, B44 |
| (117) | (54.4; 79.2) | (62.1; 85.6) | (69.4; 90.6) |
| German | A1, A2, A3 | A1, A2, A3, A24 | A1, A2, A3, A24, B44 |
| (295) | (59.9; 83.9) | (67.4; 89.4) | (72.9; 92.7) |
| Indian | A2, A11, A24 | A1, A2, A11, A24 | A1, A2, A11, A24, A33 |
| (99) | (46.1; 70.9) | (57.2; 81.7) | (66.4; 88.7) |
| USA | A1, A2, A3 | A1, A2, A3, A24 | A1, A2, A3, A24, B7 |
| (caucasoid) | (57.4; 81.9) | (67.0; 89.1) | (72.6; 92.5)^{c} |
| Japanese | A2, A24, A26 | A2, A11, A24, A26 | A2, A11, A24, A26, A31 |
| (Wajin) (1023) | (70.4; 91.2) | (80.8; 96.3) | (88.8; 98.7) |
| Chinese | A2, A11; A24 | A2, A11, A24, B13 | A1, A2, A11, A24, B13 |
| (N. Ham) (145) | (69.6; 90.8) | (75.7; 94.1) | (80.4; 96.2) |
| Chinese | A2, A11, A24 | A2, A11, A24, A33 | NC |
| (S. Ham) (138) | (85.5; 97.9) | (92.0; 99.4) | NC^{d} |
| Thais | A2, A11, A24 | A2, A11, A24, A33 | A2, A11, A24, A33, B52 |
| (242) | (72.6; 92.5) | (86.2; 98.1) | (89.3; 98.9)^{e} |

^{a}G is the genotypic and F the phenotypic coverage
^{b}Alternately, {A2, A3, A28, A30, B53} covers 54.5% of genotype or 79.3% of phenotype
^{c}Alternately, {A1, A2, A3, A11, A24} covers 72,5% of genotype or 92.4% of phenotype
^{d}Not calculated
^{e}Alternately, {A2, A11, A24, A33, B13} covers 89.1% genotype or 98.8% phenotype

TABLE 2
Peptides from Conserved Portions of the HIV-1 Genome
Some peptides from the highly conserved portions of the ENV,
GAG and POL genes of the HIV-1 genome. These peptides contain
the binding motifs for the HLA alleles indicated.

| Allele | ENV | GAG | | POL | | |
|---|---|---|---|---|---|---|
| A1 | 1 {MRDNWRSELY} | 0 {} | | 3 {NNETPGIRY LKEPVHGVY PAETOQETAY} | | |
| A2 | 7 {SLCLFSYHRL | 2 {QMREPRGSDI | | 23 | {LLDTGADDTV | KMIGGIGGFI |
| | WLWYIKIFI | EMMTACQGV} | | VLVGPTPVNI | LLTQIGCTL | TLNFPISPI |
| | QLTVWGIKQL | | | PLTEEKIKAL | QLGIPHPAGL | GLKKKKSVTV |
| | LLQLTVWGI | | | DLYVGSDLEI | ELHPDKWTV | KLLRGTKAL |
| | TLTVQARQL | | | ELELAENREI | ELAENREIL | ILKEPVHGV |
| | DMRDNWRSEL | | | QLTEAVQKI | PLVKLWYQL | ALQDSGLEV |
| | KLTPLCVTL} | | | QLIKKEKVYL | NLPPVVAKEI | SMNKELKKI |
| | | | | ELKKIIGQV | HLKTAVQMAV | LLWKGEGAV} |
| A3 | 6 {DLRSLCLFSY | 3 {GLNKIVRMY | | 23 | {KLKPGMDGPK | EMEKEGKISK |
| | IVNRVRQGY | ILDIRQGPK | | LVDFRELNK | TVLDVGDAY | WMGYELHPDK |
| | LLGIWGCSGK | LVQNANPDCK} | | KLNWASQIY | QLCKLLRGTK | ELAENREILK |
| | LLQLTVWGIK | | | ILKEPVHGVY | FVNTPPLVK | LVKLWYQLEK |
| | TLFCASDAK | | | EVNIVTDSQY | QLIKKEKVY | VLFLDGIDK |
| | TVYYGVPVWK} | | | QLDCTHLEGK | LVAVHVASGY | KLAGRWPVK |
| | | | | VVESMNKELK | QVRDQAEHLK | QMAVFIHNFK |
| | | | | AVFIHNFKRK | VVIQDNSDIK | KVVPRRKAK} |
| A11 | 5 {LLGIWGCSGK | 3 {KIRLRPGGK | | 28 | {MIGGIGGFIK | PIETVPVKLK |
| | LLQLTVWGIK | ILDIRQGPK | | KLKPGMDGPK | CTEMEKEGK | AIKKKDSTK |
| | IISLWDQSLK | LVQNANPDCK} | | LVDFRELNK | GIPHPAGLK | AIFQSSMTK |
| | TLFCASDAK | | | MTKILEPFRK | TTPDKKHQK | QLCKLLRGTK |
| | TVYYGVPVWK} | | | ELAENREILK | FVNTPPLVK | LVKLWYQLEK |
| | | | | QIIEQLIKK | IIEQLIKKEK | GIGGNEQVDK |
| | | | | VLFLDGIDK | QLDCTHLEGK | KLAGRWPVK |
| | | | | VVESMNKELK | QVRDQAEHLK | AVFIHNFKRK |
| | | | | IIATDIQTK | DIQTKELQK | VIQDNSDIK |
| | | | | DIKVVPRRK | KVVPRRKAK} | |
| A24 | 1 {IFIMIVGGL} | 3 {AFSPEVIPMF | | 6 {IFQSSMTKIL | PFLWMGYEL | VYYDPSKDL |
| | | IYKRWIILGL | | TYQIYQEPF | IYQEPFKNL | FFREDLAFL |
| | | PFRDYVDRF} | | | | |
| A28 | 3 {ISLWDQSLK | 1 {LVQNANPDCK} | | 16 {PTPVNIIGR | ISPIETVPVK | CTEMEKEGK |
| | TTLFCASDAK | | | LVDFRELNK | MTKILEPFR | GSDLEIGQHR |
| | TVYYGVPVWK} | | | TTPDKKHQK | FVNTPPLVK | LVKLWYQLEK |
| | | | | KVLFLDGIDK | ASCDKCQLK | VVESMNKELK |
| | | | | ESMNKELKK | QVRDQAEHLK | AVFIHNFKR |
| | | | | VVIQDNSDIK | NSDIKVVPR | KVVPRRKAK |
| B7 | 2 {IPIHYCAPA | 3 {SPRTLNAWV | | 16 | {TPVNIIGRNL | FPISPIETV |
| | KPCVKLTPL} | SPEVIPMFSA | | SPIETVPVKL | KPGMDGPKV | GPKVKQWPL |
| | | GPKEPFRDYV} | | WPLTEEKIKA | TPGIRYQYNV | LPQGWKGSPA |
| | | | | PPFLWMGYEL | HPDKWTVQPI | PPLVKLWYQL |
| | | | | LPPVVAKEI | PPVVAKEIV | IPAETGQETA |
| | | | | IPYNPQSQGV | VPRRKAKII} | |
| B8 | 1 {VNRVQGY} | 6 {WEKIRLRP | | 22 | {FIKVRQYD | PVKLKPGM |
| | | KIRLRPGG | | GPKVKQWPL | MEKEGKISK | EGKISKIGP |
| | | QMREPRGSD | | AIKKKDST | STKWRKLV | GLKKKKSV |
| | | LNKIVRMYS | | LKKKKSVT | DKKHQKEPP | LCKLLRGTK |
| | | NCRAPRKKG | | LLRGTKALT | PFKNLKTGK | NLKTGKYAR |
| | | APRKKGCW} | | ANRETKLGK | ETKLGKAGY | IKKEKVYL |
| | | | | MNKELKKII | NFKRKGGI | VPRRKAKI |
| | | | | RRKAKIIR | KAKIIRDYG | |
| B27 | 2 {FRPGGGDMR | 3{IRLRPGGKK | PRTLNAWVK | 3 {VRDQAEHLK | FRVYYRDSR | PRRKAKIIR} |
| | WRSELYKYK} | KRWIILGLNK | IRQGPKEPFR | | | |
| | | ERQANFLGK | | | | |

| | | | | | |
|---|---|---|---|---|---|
| B35 | 0 {} | 1 {GPKEPFRDY} | 4 | {TPGIRYQY | PPFLWMGY |
| | | | EPVHGVYY | PPLVKLWY} | |
| B40 | 1 {IEAQQHLLQL} | 4 {PEVIPMFSA | 9 {SEQTRANSPT | KEALLDTGA | YELHPDKWT |
| | | SEGATPQDL | LELAENREIL | QEPFKNLKT | WEFVNTPPL |
| | | REPRGSDIA | AETFYVDGA | HEKYHSNWRA | IEAEVIPAET} |
| | | TETLLVQNA} | | | |

### EXAMPLE 3

In this example, Peptide Selection for HIV-1 conjugates are described. The procedure described above has been followed through the selection of conserved portions of the HIV-1 genome which encode peptides corresponding to the binding motifs for a set of 5 HLA alleles covering over 90% of the Caucasian population in the USA. 114 possible peptide-allele pairs were identified, and the peptides are listed on Table 2.

This invention contemplates selecting at least one peptide from the group of peptides in Table 2 which bind to HLA allele A1; at least one peptide selected from the group of peptides in Table 2 which bind to HLA allele A2; and at least one peptide selected from the group of peptides in Table 2 which bind to HLA allele A3. Preferably, the target peptide would be selected from the group of peptides in Table 2 which bind to HLA allele A24, and more preferably selected from the group of peptides in Table 2 which bind to HLA allele B7. The peptide selected will bind to HLA alleles present in at least 80% of the Caucasian population in many geographic areas, based on the frequencies reported in Table 1.

As shown in Table 3, 6 cytotoxic T cell epitopes (from the Los Alamos HIV database) are presented by HLA-A2 molecules on cells from patients with AIDS. Analysis of HIV strains indicates that they are all highly conserved. The first column lists the HIV protein from which they are derived: the second gives their position in the protein; the third is the actual peptide sequence. Table 4 lists all the reported CTL epitopes for HIV. These are from AIDs patients and are taken from the Los Alamos database.

Thus, antibodies can be generated to the selected target MHC-peptide complex and then conjugated to an appropriate cytotoxic agent as delineated in the Detailed Description section.

TABLE 3

| | Position | Sequence |
|---|---|---|
| p24 | (19-27) | TLNAWVKVI |
| | | TLNAWVKVV |
| RT | (263-273) | VLDVGDAYFSV |
| RT | (334-342) | VIYQYMDDL |
| RT | (464-472) | ILKEPVHGV |
| RT | (640-648) | ALQDSGLEV |
| RT | (956-964) | LLWKGEGAV |

TABLE 4

| Presented | | | |
|---|---|---|---|
| by | Known motif | Protein | |
| HLA-A2 | x[LM]xxxxxx[VLI] | p17 (69-93) | QTGSEBLRSLYNTVATLYCVHQRIE |
| | | 3X P17 (77-85) | SLYNTVATL |
| | | P17 (88-115) | VHQRIEIKDTKEALDKIEEEQNKSKKKA |
| | | P24 (11-32) | VHQAISPRTLNAWVKVVEEKAF |
| | | P24 (19-27) | TLNAWVKVV |
| | | P24 (61-71) | QHQAAMQMLKE |
| | | P24 (87-101) | HAGIAPGQMREPRG |
| | | P15 (69-83) | GNFLQSRPEPTAPPF |
| | | P15(41-56) | KEGHQMKDCTERQANF |
| | | RT(263-273) | VKDVDAYFSV |
| | | RT(334-342) | VIYQYMDDL |
| | | RT(449-473) | PLTEEAELELAENREILKEPVHGVY |
| | | 4X RT(464-472) | ILKEPVHGV |
| | | RT(640-648) | ALQDSGLEV |
| | | 2X RT(956-964) | LLWKGEGAV |
| | | GP120(32-41) | KLWVTVYYGV |
| | | GP120(33-54) | LWVTVYYGVPVWKEATTTLFCA |
| | | GP120(104-116) | HEDIISLWDQSLK |
| | | GP120(120-128) | KTLPLCVTL |
| | | GP120(192-211) | TTSYTLTSCNTSVITQACPK |
| | | GP120(196-204) | KLTSCNTSV |
| | | | TLTSCNTSV |
| | | GP120(295-311) | SVEINCTRPNNNTRKSI |
| | | 3X 310-324 | RIQRGPGRAFVTIGK |
| | | 373-379 | PEIVTHS |
| | | 380-391 | NSGGEFFYSNS |
| | | 380-391 | KNCGGEFFYCNS |
| | | 417-436 | LPCRIKQFINMWQEVGKAMY |
| | | 422-436 | KQFINMWQEVGKAMY |
| | | 422-437 | |
| | | 422-437 | |
| | | 491-510 | VKIEPLGVAPTKAKRRVVQR |
| | | GP41 (237-245) | RLVNGSLAL |
| | | 303-312 | SLLNATDIAV |
| | | 304-312 | |
| | | 317-331 | DRVIEVVQGAYRAIR |
| | | 318-326 | RVIEVLQRA |
| | | NEF (71-80) | QVPLRPMTYK |
| | | 84-98 | DLSHFLKEKGGLEGL |
| | | 178-187 | VLEWRFDSRL |
| | | 188-196 | AFHHVAREL |

USA CAUCASIAN

| | | | |
|---|---|---|---|
| HLA-A1 | XX[ED]XXXXXY | NEF(180-196) | EWRFDSRLAFHHVAREL |
| HLA-A3 | X[LMV]XXXXXX[KY] | GP120(310-324) | RIQRGPGRAFVTIGK |
| HLA-A24 | X[YF]XXXXXX[FL] | P17(28-36) | KYKLKHIVW |
| | | GP41(76-83) | YLKDQQLL |
| | | NEF(118-142) | YFPDWQNYTPGPGIRYPLTFGWCYK |
| HLA-B7 | X[LAVI] | | |

NORTH AMERICAN NEGROID

| | | |
|---|---|---|
| HLA-28 | RT 86-94 | DTVLEEMNL |
| | 519-527 | DVKQLTEVV |
| | 596-602 | AETFYVDGAAN |
| HLA-A30 | | |
| HLA-A33 | | |
| HLA-B53 | | |

DISTRIBUTION for 68 ctl epitopes

| | |
|---|---|
| unknown | 1 |
| A2 | 47 |
| A3 | 2 |
| A24 | 3 |
| A26 | 2 |
| A25 | 4 |
| A28 | 3 |
| A29 | 2 |
| A*6802 | 1 |
| B27 | 1 |
| B38 | 1 |
| B52 | 1 |

### EXAMPLE 4

In this example, Peptide Selection for Papillomavirus-conjugates are described. The following information is from a public database (maintained by Professor T. Smith) in the Department of Biomedical Engineering at Boston University. Papillomavirus strain 16 has been implicated in the etiology of cervical cancer. Seven conserved peptide sequences from Papilloma strain 16, proteins E6 and E7 that were identified to bind HLA-A2, are listed on Table 5. The column after type indicates the name of the strain (11, 16 etc), followed by the name of the protein (E6 or E7); the protein fragment (e.g., residues 4-11); and the sequence of the fragment, using single letter code for the amino acid (e.g., R=Arginine; L=Leucine; V=Valine etc.). The astericks indicated by ** were found to be conserved. These peptides conjugated to HLA-A2 were the initial targets against which high affinity antibodies can be raised. Since, HLA-A2 is present over 40% of the population, such antibodies can serve as diagnostics for a large percentage of the population. When conjugated to an appropriate toxin, they would serve as therapeutics. Likewise, identification of peptides that bind other HLA alleles can be similarly determined.

TABLE 5
Herpes Papilloma A2 Binding Peptides

Papillamavirus type 11 E7 ( 4-11) RLVTLKDIV
Papillomavirus type 16 E7 ( 7-15) TLHEYMLDL **
Papillomavirus type 16 E7 (11-20) YMLDLQPETT
Papillamavirus type 11 E7 (22-30) GLHCYEQLV
Papillamavirus type 6B E7 (47-55) PLKQHFQIV
Papillomavirus type 16 E7 (66-74) RLCVQSTHV **
Papillomavirus type 16 E7 (82-90) LLMGTLGIV **
Papillomavirus type 16 E7 (86-93) TLGIVCPI **
Papillamavirus type 16 E6 ( 7-15) AMFQDPQER
Papillamavirus type 16 E6 (18-26) KLPQLCTEL **
Papillomavirus type 16 E6 (26-34) LQTTIHDII **
Papillamavirus type 16 E6 (29-38) TIHDIILECV **
Papillamavirus type 16 E6 (52-60) FAFRDLCIV

What is claimed is:

1. A method of constructing targeting antigens,
a) providing:
i) sequenced genomes of multiple variants of a pathogen, and
ii) class I MHC molecules which occur with greatest frequency in a population of interest;
b) identifying conserved regions of said genomes, said conserved regions encoding peptides;
c) determining which of said peptides bind to said class I MHC molecules, thereby selecting MHC-binding peptides and corresponding class I MHC molecules; and
d) constructing targeting antigens comprised of one or more of said MHC-binding peptides bound to said corresponding class I MHC molecule.

2. The method of claim 1, wherein said pathogen is a virus.

3. The method of claim 2, wherein said virus is HIV.

4. The method of claim 1, wherein said pathogen is bacterial.

5. The method of claim 1, wherein said MHC-binding peptide is a peptide variant.

6. An antibody directed against the targeting antigen constructed according to claim 1.

7. A method of producing an antibody, comprising:
a) providing:
i) a targeting antigen comprised of one or more MHC-binding peptides bound to a corresponding class I MHC molecule and
ii) a host for immunization; and
b) immunizing said host with said antigen, under conditions such that an antibody is produced.

8. The method of claim 7, wherein said antibody produced is a polyclonal antibody.

9. The method of claim 7, wherein said antibody produced is a monoclonal antibody.

10. A method of conjugating an antibody, comprising:
a) providing:
i) an antibody directed against a targeting antigen, said antigen comprising one or more MHC-binding peptides bound to a corresponding class I MHC molecule and
ii) a toxic agent; and
b) conjugating said toxic agent to said antibody under conditions such that a conjugated antibody is produced.

11. The method of claim 10, wherein said antibody is a polyclonal antibody.

12. The method of claim 10, wherein said antibody is a monoclonal antibody.

13. The method of claim 10, wherein said toxic agent is ricin or a cytotoxic portion thereof.

14. The method of claim 10, wherein said toxic agent is radioactive.

15. A composition, comprising an antibody directed against a targeting antigen, said antigen comprising one or more MHC-binding peptides bound to a corresponding class I MHC molecule, wherein said antibody is conjugated to a toxic agent.

16. The composition of claim 15, wherein said antibody is a monoclonal antibody.

17. The composition of claim 15, wherein said antibody is a polyclonal antibody.

18. The composition of claim 15, wherein said toxic agent is ricin or a cytotoxic portion thereof.

19. The composition of claim 15, wherein said toxic agent is radioactive.
